(19) United States
(12) Patent Application Publication
(10) Pub. No.: US 2019/0300458 A1
(43) Pub. Date: Oct. 3, 2019

(54) METHODS FOR PREPARING CHLORINATED ALKANES BY UTILIZING FERRIC CHLORIDE, AN INITIATOR, AND AN ALKYLPHOSPHATE

(71) Applicant: BLUE CUBE IP LLC, Clayton, MO (US)

(72) Inventors: Max Tirtowidjojo, Clayton, MO (US); Marc Sell, Clayton, MO (US); John D. Myers, Clayton, MO (US)

(21) Appl. No.: 16/373,221

(22) Filed: Apr. 2, 2019

Related U.S. Application Data
(60) Provisional application No. 62/652,075, filed on Apr. 3, 2018.

(51) Int. Cl. C07C 17/02; C07C 17/04

(52) U.S. Cl. CPC C07C 17/02; C07C 17/04

(57) ABSTRACT

The present invention provides processes for the production of chlorinated alkanes.

### CROSS REFERENCE TO RELATED APPLICATIONS

The instant application claims the benefit of U.S. Provisional Patent Application Ser. No. 62/652,075, filed Apr. 3, 2018, which is incorporated herein by reference in its entirety.

### FIELD OF THE INVENTION

The present disclosure generally relates to processes for preparing chlorinated alkanes.

### BACKGROUND OF THE INVENTION

Halogenated alkanes are useful intermediates for many products including agricultural products, pharmaceuticals, cleaning solvents, blowing agents, solvents, gums, silicones, and refrigerants. The processes to prepare halogenated alkanes can be time consuming, moderately efficient, and lack reproducibility.

One widely known method for preparing halogenated alkanes is through a telomerization process. This process comprises contacting carbon tetrachloride and an alkene or halogenated alkene in the presence of a catalyst. Even though these telomerization processes are useful, these processes have inconsistent yields, low reproducibility, large amounts of waste, and high unit manufacturing costs.

One subset of highly sought halogenated alkanes are chloropropanes especially 1,1,1,3-tetrachloropropane and 1,1,1,3,3-pentachloropropane, which are useful intermediates for many products, including refrigerants and agricultural products. A general process for their preparation consists of reacting an alkene or a halogenated alkene, carbon tetrachloride, a ligand or promoter such as a trialkylphosphate, and an iron catalyst. U.S. Pat. No. 4,650,914 teaches such a process where the process is conducted in batch mode, using a non-powder form of an iron (Fe(0)) and mechanical stirring. All materials are introduced into an autoclave wherein the ethylene is added to pressurize the autoclave. US 2004/0225166 teaches a similar process using a single reactor in a continuous process. Ethylene is fed into the reactor comprising carbon tetrachloride, tributylphosphate, and iron powder. The reactor is pressurized from 40 to 200 psi to maintain a concentration of ethylene. Similarly, iron (Fe(0)) utilized as a solid in these processes must undergo an oxidation and/or reduction to form the active, soluble catalytic species necessary to initiate the telomerization process.

These conventional processes can be moderately efficient yet lack reproducibility, utilize expensive manufacturing equipment, have large waste factors, require heterogeneous reaction mixtures, rely upon complex and unpredictable solid-liquid catalysis and mass transfer and/or provide the chlorinated propane at a higher unit manufacturing cost.

Developing a process which can prepare halogenated alkanes and chlorinated propanes where the process would exhibit high reproducibility, high selectivity, reduced amounts of waste, and reduced manufacturing costs would be desirable.

### SUMMARY OF THE INVENTION

In one aspect, provided herein are processes for preparing chlorinated alkanes via a reaction between an alkene, chlorinated alkene, or combinations thereof and carbon tetrachloride. The processes comprise preparing a homogeneous, liquid phase reaction mixture comprising carbon tetrachloride (Tet), an alkene, a chlorinated alkene, or combinations thereof, a liquid phase free radical initiator, optionally a ligand, and a transition metal salt. Once the homogeneous, liquid phase reaction mixture is prepared, a product mixture comprising the chlorinated alkane, light by-products, and heavy by-products is produced. Separating the chlorinated alkane from the product mixture provides the chlorinated alkane in high yield.

In another aspect, provided herein are processes for the preparation of 1,1,1,3-tetrachloropropane. The processes comprise preparing a homogeneous, liquid phase reaction mixture comprising carbon tetrachloride, ethylene, a liquid phase free radical initiator, and a transition metal salt. Once the homogeneous, liquid phase reaction mixture is prepared, a product mixture comprising 1,1,1,3-tetrachloropropane, light by-products, and heavy by-products is produced. Separating the 1,1,1,3-tetrachloropropane from the product mixture provides the 1,1,1,3-tetrachloropropane in high yield.

In a further aspect, provided herein are processes for the preparation of 1,1,1,3,3-pentachloropropane. The processes comprise preparing a homogeneous, liquid phase reaction mixture comprising carbon tetrachloride, vinyl chloride, a liquid phase free radical initiator, and a transition metal salt. Once the homogeneous, liquid phase reaction mixture is prepared, a product mixture comprising 1,1,1,3,3-pentachloropropane, light by-products, and heavy by-products is produced. Separating the 1,1,1,3,3-pentachloropropane from the product mixture provides the 1,1,1,3,3-pentachloropropane in high yield.

Other features and iterations of the invention are described in more detail below.

### BRIEF DESCRIPTION OF FIGURES

FIG. 1 is a graphical representation of the % conversion of carbon tetrachloride to 1,1,1,3-tetrachloropropane versus run time.

FIG. 2 is a graphical representation of the % selectivity of carbon tetrachloride to 1,1,1,3-tetrachloropropane versus the % conversion of carbon tetrachloride to 1,1,1,3-tetrachloropropane.

### DETAILED DESCRIPTION OF THE INVENTION

Disclosed herein are processes for the preparation of chlorinated alkanes. In general, the processes comprise a reaction between an alkene, a chlorinated alkene, or combinations thereof and carbon tetrachloride under conditions described herein.

A homogeneous liquid phase reaction mixture is prepared by contacting an alkene, a chlorinated alkene, or combinations thereof, carbon tetrachloride, a liquid phase free radical initiator, and a transition metal salt. Once the homogeneous liquid phase reaction mixture is prepared, a product mixture comprising light by-products, heavy by-products, and the chlorinated alkane is produced.

The processes, described below, show the synergistic effect that a liquid free radical initiator and a transition metal salt have on improving selectivity and yield of the chlorinated alkane. Additionally, the % conversion of the carbon tetrachloride to the chlorinated alkane is demonstrated using a transition metal salt and a liquid free radical initiator. The results in regards to improvements in selectivity, yield, and % conversion are surprising and unexpected.

An additional aspect of the present invention, separating the chlorinated alkane from the homogeneous, liquid phase reaction mixture and recycling product effluent streams back to the reactor, is also described herein. In this aspect, the product effluent stream that is recycled to the reactor comprises less chlorinated alkane when compared to the homogeneous, liquid phase reaction mixture. The recycling of product effluent streams back into the process provides added efficiency and cost reduction of the process.

### (I) Processes to Prepare Chlorinated Alkanes

One aspect of the present disclosure encompasses processes for the preparation of chlorinated alkanes. The process comprises forming a homogeneous liquid phase reaction mixture comprising carbon tetrachloride (Tet), an alkene, a chlorinated alkene, or combinations thereof, a liquid phase free radical initiator, and a transition metal catalyst. Once the homogeneous liquid phase reaction mixture is prepared, the chlorinated alkane, light by-products and heavy by-products are formed. The process is conducted to a selectivity of at least 50%. After the separation of light by-products, heavy by-products, the chlorinated alkane is produced in high yield and at a reduced manufacturing cost.

(a) An Alkene, a Chlorinated Alkene, or Combinations Thereof

An alkene, a chlorinated alkene, or combinations thereof is used in the process. As appreciated by the skilled artisan, the alkene, a chlorinated alkene, or combinations thereof may be introduced in the reaction as a gas or a liquid wherein the alkene, a chlorinated alkene, or combinations thereof may be at least partially soluble in the liquid phase. Additionally, the alkene, chlorinated alkene, or combinations thereof may undergo a phase transition from a liquid to a gas wherein a portion of the gas occupies the headspace of the reactor. In various embodiments, the alkene, a chlorinated alkene, or combinations thereof may be introduced above the surface of the liquid phase or below the surface of the liquid phase through a port in the reactor. The alkene, a chlorinated alkene, or combinations thereof may be introduced into the reactor to prepare a high concentration of the alkene, a chlorinated alkene, or combinations thereof in carbon tetrachloride. Additionally, the alkene, a chlorinated alkene, or combinations thereof may be added during the process to maintain a pressure within the reactor.

Generally, the at least one alkene, chlorinated alkene, or combinations thereof comprise between 2 and 5 carbon atoms. Non-limiting examples of alkenes may be ethylene, propylene, 1-butene, 2-butene, isobutene, 1-pentene, 2-pentene, 3-pentene, 2-methyl-2-butene, 2-methyl-1-butene, and 3-methyl-1-butene. Non-limiting examples of chlorinated alkenes may be vinyl chloride, allyl chloride, vinylidene chloride, 1-chloro-2-butene, 3-chloro-1-butene, 3-chloro-1-pentene, trichloroethylene, perchloroethylene, 1,2,3-trichloropropene, 1,1,3-trichloropropene, 3,3,3-trichloropropene and combinations thereof. In one preferred embodiment, the alkene comprises ethylene. In another preferred embodiment, the halogenated alkene is a chlorinated alkene that comprises vinyl chloride monomer.

(b) Carbon Tetrachloride

Carbon tetrachloride is used in the process. In general, the carbon tetrachloride may be used in excess. Generally, the molar ratio of carbon tetrachloride to the alkene, the chlorinated alkene, or combinations thereof may range from 0.1:1 to about 100:1. In various embodiments, the molar ratio of the carbon tetrachloride to the alkene, the chlorinated alkene, or combinations thereof may range from 0.1:1 to about 100:1, from 0.5:1 to about 75:1, from 1:1 to about 10:1, or from 1.2:1 to about 5:1. In various embodiments, the molar ratio of carbon tetrachloride to the alkene, the chlorinated alkene, or combinations thereof may range from 1.2:1 to about 2:1. Carbon tetrachloride and the alkene, a chlorinated alkene, or combinations thereof are essentially dry, i.e., it has a water content of the below about 1000 ppm. Lower water concentrations are preferred, but not required.

(c) Liquid Free Radical Initiator

A liquid free radical initiator is utilized in the process. As appreciated by the skilled artisan, the liquid free radical initiator may be a liquid in its normal physical state or may be a solid dissolved in carbon tetrachloride. Generally, the liquid free radical initiator may be an organic free radical initiator which has solubility in carbon tetrachloride. In an embodiment, the free radical initiator comprises an azo compound, a peroxide, ultraviolet light, or combinations thereof. Non-limiting examples of suitable organic or inorganic free radical initiators may include azobisisobutyronitrile, 1,1′-azobis(cyclohexanecarbonitrile), 2,2′-azobis(2-methylpropionitrile, di-tert-butylperoxide, tert-butyl peracetate, tert-butyl peroxide, methyl ethyl ketone peroxide, acetone peroxide, cyclohexane peroxide, 2,4-pentanedione peroxide, or combinations thereof.

In general, the molar ratio of the liquid free radical initiator to carbon tetrachloride may range from about 1:10 to about 1:100000. In various embodiments, the molar ratio of the liquid free radical initiator to carbon tetrachloride may range from 1:10 to about 1:100000, from 1:100 to about 1:10000, from 1:500 to about 1:5000, or from about 1:750 to about 1:1000.

(d) Transition Metal Salt

A wide variety of transition metal salts may be used in the process. Non-limiting examples of suitable transition metal salts may be an aluminum salt, a bismuth salt, a chromium salt, a cobalt salt, a copper salt, a gallium salt, a gold salt, an indium salt, an iron salt, a lead salt, a magnesium salt, a manganese salt, a mercury salt, a nickel salt, a platinum salt, a palladium salt, a rhodium salt, a samarium salt, a scandium salt, a silver salt, a titanium salt, a tin salt, a zinc salt, a zirconium salt, or combinations thereof. In an embodiment, suitable transition metal salts which may be used in the process may be an iron salt or a copper salt. In an embodiment, the transition metal salt is an iron salt. Non-limiting examples of suitable transition metals salts may be copper (I) salt, copper (II) salt, iron (II) salt, and iron (III) salt. In an embodiment, the iron salt comprises iron (II) chloride, iron (III) chloride, or combinations thereof. As appreciated by the skilled artisan, a wide variety of anions may be part of the transition metal salt. Non-limiting examples of suitable anions in the transition metal salts may include acetates, acetyacetonates, alkoxides, butyrates, carbonyls, dioxides, halides, hexonates, hydrides, mesylates, octanoates, nitrates, nitrosyl halides, nitrosyl nitrates, sulfates, sulfides, sulfonates, phosphates, and combinations thereof. In a preferred embodiment, the anion in the transition metal salt comprises a chloride. In a preferred embodiment, the transition metal salt may be copper (I) chloride, copper (II) chloride, iron (II) chloride, iron (III) chloride, or combinations thereof.

As appreciated by the skilled artisan, the transition metal salt, once in the process, may undergo oxidation and/or reduction to produce an activated catalytic species in various oxidation states. The oxidation state of these active iron catalytic species may vary, and may be for examples (I), (II), and (III). In an embodiment, the active iron catalyst may in the Fe(I) oxidation state. In another aspect, the active iron catalyst may be Fe(II). In still another aspect, the active iron catalyst may be in the Fe(III) oxidation state. In an additional aspect, the active iron catalyst may comprise a mixture of Fe(I) and Fe(II). In still another aspect, the active iron catalyst may comprise a mixture of Fe(I) and Fe(III) oxidation states. In yet another aspect, the active iron catalyst may be in the Fe(II) and Fe(III) oxidation states. In another aspect, the active iron catalyst may in the Fe(I), Fe(II) and Fe(III) oxidation states. In still another embodiment, an electrochemical cell may be utilized to adjust the ratio of Fe(I), Fe(II), and Fe(III) in the process. The oxidation state of these active copper catalytic species may vary, and may be for examples, (I), and (II). In one aspect, the active copper catalyst may be Cu(I). In still another aspect, the active copper catalyst may be in the Cu(II) oxidation state. In an additional aspect, the active copper catalyst may comprise a mixture of Cu(I) and Cu(II). In still another aspect, an electrochemical cell may be utilized to adjust the ratio of Cu(I), and Cu(II) in the process.

Generally, the molar ratio of the carbon tetrachloride to the transition metal salt may range from about 10,000:1 to about 1:1. In various embodiments, the molar ratio of the carbon tetrachloride to the transition metal salt may range from 10,000:1 to about 1:1, from 5,000:1 to about 10:1, from 500:1 to about 50:1, or from 125:1 to about 75:1.

(e) Ligand

In various embodiments, the transition metal catalyst further comprises at least one ligand. The ligand, as the skilled artisan appreciates, may form a complex with the transition metal salt forming a transition metal ligand containing compound complex which is soluble within the reaction media. Examples of ligand containing compound may include trialkylphosphates, trialkylphosphites, C₃-C₆ alkane nitrile, or combinations thereof.

In one embodiment, the ligand is a phosphorus containing compound. Examples of phosphorus containing compound may include trialkylphosphates, trialkylphosphites, or combinations thereof. Suitable non-limiting examples of trialkylphosphates include triethylphosphate, tripropylphosphate, triisopropylphosphate, tributylphosphate, or combinations thereof. Non-limiting examples of trialkylphosphites include trimethylphosphite, triethylphosphite, tripropylphosphite, triisopropylphosphite, tributylphosphite, tri-tertbutylphosphite or combinations thereof. In one preferred embodiment, the phosphorus containing compound is a trialkylphosphate, namely tributylphosphate.

In another embodiment, the ligand is a C₃-C₆ alkanenitrile. Non-limiting examples of C₃-C₆ alkanenitrile may be propanenitrile, butanenitrile, pentanenitrile, hexanenitrile, or combinations thereof.

Generally, the molar ratio of the transition metal salt to the ligand may range from 1:1 to about 1:1000. In various embodiments, the molar ratio of the transition metal salt to the ligand may range from 1:1 to about 1:1000, from 1:1 to about 1:500, from 1:1 to about 1:100, or from 1:1 to about 1:10.

(f) Optional Use of UV Light

In various embodiments, UV light may be used to enhance the reaction. In general, the exposure of UV light to the reaction may occur for a period of a few minutes or throughout the entire process.

(g) Reaction Conditions

As appreciated by the skilled artisan, there are many methods to stir the contents of a reactor and/or provide increased gas absorption into the liquid phase. These methods would provide a high concentration the alkene, chlorinated alkene, or combinations thereof in carbon tetrachloride. In various embodiments, these methods simply mix the liquid phase of the reaction mixture, such as mechanical stirring. In other embodiments, the method not only mixes the liquid phase of the reaction mixture but also provide increased gas absorption into the liquid phase of the reaction mixture. In still another embodiment, the method provides increased absorption of the gas phase into the liquid phase of the reaction mixture of the reactor. Non-limiting methods to adequately stir the liquid phase contents of the reactor may be jet stirring, mechanical stirring using impellers, mechanical stirring using impellers and baffles in the reactor, or combinations thereof. In an embodiment, the homogeneous liquid phase reaction mixture is stirred and stirring the homogeneous liquid phase reaction mixture comprises mechanical stirring, stirring by liquid jet mixing, or combinations thereof. Non-limiting examples of methods to not only mix the contents of the reactor but also provide increased gas absorption into the liquid phase reaction mixture may be jet stirring using at least one eductor, jet stirring comprising at least one nozzle and at least one eductor, jet stirring wherein jet stirring comprises at least one nozzle is directed through the gas phase into the liquid phase, specially designed impellers which create adequate gas absorption into the liquid phase, reactors with specially designed baffles, and combinations thereof. A non-limiting example of a method to provide increased absorption of the gas phase into the liquid phase of a reactor may be a spray nozzle wherein the liquid phase reaction mixture is pumped through the spray nozzle into the gas phase resulting in absorption of the gas into the liquid spray. At least one of these methods may be utilized in the process to maintain the kinetic of the process.

Jet mixing utilizing at least one nozzle, as appreciated by the skilled artisan, withdraws a portion of the liquid phase of the reaction mixture from the reactor and pumps the liquid phase back into the reactor through at least one nozzle, thereby creating turbulence in the liquid phase. The at least one nozzle may be positioned below the surface of the liquid phase, thereby creating turbulence in the liquid phase and providing increased mixing. The at least one nozzle may be positioned at the surface of the liquid phase or directed through the gas phase into the liquid phase, thereby providing increased turbulence of the reaction mixture but also provides increased absorption of the gas phase into the liquid phase.

Jet mixing utilizing at least one eductor, as appreciated by the skilled artisan, withdraws a portion of the liquid phase of the reaction mixture from the reactor and pumps the liquid phase back into the reactor through at least one gas educting nozzle. The eductor nozzle provides suction in the eductor which pulls gas from the gas phase of the reaction mixture, mixes the gas with the circulated liquid phase, and returns the resulting mixture of liquid and gas back into the liquid phase of the reactor where the liquid had increased absorption of the gas as compared to the circulated liquid phase. When the flow from the eductor nozzle is directed towards the liquid phase of the reaction mixture, increased gas absorption of the gas in the liquid phase and increased turbulence of the reaction mixture result.

Jet mixing may also utilize at least one nozzle and at least one eductor. In this configuration, as described above, not only increased turbulence in the reaction mixture but also increased gas absorption of the gas into the liquid phase may be realized.

The use of a spray nozzle may also be utilized. Using a spray nozzle, the liquid phase is pumped through the spray nozzle producing droplets of the liquid phase from the reaction mixture. These droplets may be discharged into the gas phase, where they absorb at least some of the gas phase. The droplets are then reincorporated into the liquid phase of the reaction mixture, thereby increasing the amount of gas dissolved in the liquid phase of the reaction mixture.

In other embodiments, the homogeneous liquid phase reaction mixture is formed in a reactor and the reactor comprises a draft tube. The draft tube provides an internal recirculation of the reaction mixture. The circulation may be induced by energy from the at least one liquid jets, from the at least one gas educting nozzle, from rising gas bubbles within the reactor, or a combination thereof.

As appreciated by the skilled artisan, at least one of the methods or a combination of these may be utilized in the process.

In general, the process for the preparation of halogenated alkanes will be conducted to maintain the temperature from about 80° C. to about 140° C. using an internal or external heat exchanger. In various embodiments, the temperature of the reaction may be maintained from about 80° C. to about 140° C., from 85° C. to about 125° C., from 90° C. to about 120° C., or from about 95° C. to about 110° C.

Generally, the process may be conducted at a pressure of about atmospheric pressure (˜14.7 psi, 101.3 kPa) to about 200 psi (1379 kPa) so the amount of the gases and liquid are in suitable quantities so the reaction may proceed and maintain the kinetics of the process. In various embodiments, the pressure of the process may be from about atmospheric pressure (˜14.7 psi, 101.3 kPa) to about 200 psi (1379 kPa), from about 20 psi (137.9 kPa) to about 180 psi (1241 kPa), from about 40 psi (275.8 kPa) to about 160 psi (1103.1 kPa), from about 80 psi (551.6 kPa) to about 140 psi (965.3 kPa), or from 100 psi (689.5 kPa) to about 120 psi (827.4 kPa).

(h) Output from Process

The process, as outlined above, produces chlorinated alkane, light by-products, and heavy by-products. In general, the process produces the chlorinated alkane in at least 95% selectivity. In various embodiments, the chlorinated alkane is produced in a selectivity of at least 95%, in at least 96%, in at least 97%, in at least 98%, in at least 99%, or in at least 99.5%.

In general, the process converts carbon tetrachloride to the chlorinated alkane in a conversion of at least 50% of the carbon tetrachloride. The skilled artisan understands the percent conversion (% conversion) is determined by the percentage of carbon tetrachloride converted to the chlorinated alkane in the product from the reaction. It is distinct from the overall conversion from the process, in which unconverted carbon tetrachloride can be recycled to effect overall process conversion up to 100%. In various embodiments, the % conversion from the reaction is at least 50%, at least 60%, is at least 70%, is at least 80%, is at least 90%, or even at least 99%.

In general, the process produces the chlorinated alkane is at least 50 weight percent (wt %) in the reaction mixture of the reactor. In various embodiments, the chlorinated alkane is produced in at least 50 wt %, in at least 60 wt %, in at least 70 wt %, in at least 80 wt %, in at least 90 wt %, in at least 95 wt %, or in at least 99 wt % in the reaction mixture of the reactor.

### (II) Separation of Chlorinated Alkane and Recycle Streams

Another aspect of the disclosure provides processes for the separation and recycle streams of the chlorinated alkane from the homogeneous, liquid phase reaction mixture comprising chlorinated alkane, the alkene, chlorinated alkene, or combinations thereof, carbon tetrachloride, the liquid free radical initiator, the at least one metal salt, optionally a ligand, light by-products, and heavy by-products.

The next step in the process comprises separating chlorinated alkane from the contents of the homogeneous, liquid phase reaction mixture in a reactor comprising carbon tetrachloride, the alkene, chlorinated alkene, or combinations thereof, liquid phase free radical initiator, transition metal catalyst, heavy by-products, and light by-products through at least one separator and alternatively a second separator in order to isolate the chlorinated alkane in the desired yield and/or purity. In various embodiments, the at least one of the first separator and the second separator may be a distillation column or a multistage distillation column. Additionally, the at least one of the first separator and the second separator may further comprise a reboiler, a bottom stage, or a combination thereof. Various distillation columns may be used in this capacity. In one embodiment, a side draw column or a distillation column which provides outlet stream from an intermediate stage or a dividing wall column (dividing wall column (DWC) is a single shell, fully thermally coupled distillation column capable of separating mixtures of three or more components into high purity products (product effluent streams) may be used as a separator where the product effluent streams comprise the chlorinated alkane, the alkene, chlorinated alkene, or combinations thereof, carbon tetrachloride, the liquid free radical initiator, the at least one metal salt, optionally a ligand, light by-products, heavy by-products, or combinations thereof. A portion of various product effluent streams produced by the process may be recycled back into the reactor to provide increased kinetics, increased efficiencies, reduced overall cost of the process, increased selectivity of the desired halogenated alkane, and increased yield of the desired halogenated alkane.

As appreciated by the skilled artisan, each product effluent stream, as described below, is enriched in the particular component of the homogeneous, liquid phase reaction mixture. Further separation may be required of each product effluent streams to produce highly pure compounds.

As appreciated by the skilled artisan, separating the chlorinated alkane from the contents of the reactor would produce at least two product effluent streams. In various embodiments, separating the purified chlorinated alkane may produce three, four, five, or more product effluent streams depending on the separation device utilized. As an example, the separation of the chlorinated alkane from the contents of the reactor using three product effluent streams is shown below.

The process utilizing at least one separator commences by transferring at least a portion of or the homogeneous, liquid phase reaction mixture of the reactor into the separator. In this operation, a portion of the homogeneous, liquid phase reaction mixture of the reactor may be separated into three distinct product effluent streams, product effluent stream (a), (b), and (c). Product stream (a) as an overhead stream comprising light by-products, the alkene, chlorinated alkene, or combinations thereof, and carbon tetrachloride; product stream (b) comprising the chlorinated alkane; and product stream (c) as a bottom stream comprising heavy by-products, the optional ligand, the liquid free radical initiator, and the transition metal salt.

In another embodiment, product effluent stream (a) may be transferred into a second separator producing two distinct product effluent streams (d) and (e). Product effluent stream (d) comprising the alkene, chlorinated alkene, or combinations thereof and light by-products while product effluent stream (e) comprises carbon tetrachloride. A portion of product effluent streams (d) comprising light by-products and the alkene, chlorinated alkene, or combinations thereof and/or product effluent stream (e) comprising carbon tetrachloride may be recycled back to the reactor.

In yet another embodiment, product effluent stream (b) comprising the chlorinated alkane may be transferred into an additional separation device to achieve the desired purity of the halogenated alkane.

In still another embodiment, a portion of product effluent stream (c) comprising heavy by-products, the at least one ligand, the liquid free radical initiator, and the transition metal salt may be recycled back to the reaction vessel, used in another process, purged to waste, or combinations thereof.

In various embodiments, at least a portion of product effluent streams (c), (d), and/or (e) may be recycled back into the reactor or mixed with fresh feed (which contains one or more of carbon tetrachloride, an alkene, chlorinated alkene, or combinations thereof, a liquid phase free radical initiator and a transition metal salt, wherein the components of the fresh feed are not recycled) before being recycled back into the reactor. These streams may also be fed into another process to produce other products. These steps may be performed in order to improve the efficiency, reduce the cost, reduce contaminants, and increase through-put of the process. In an embodiment, after at least some chlorinated alkane is formed, fresh feed comprising carbon tetrachloride; an alkene, chlorinated alkene, or combinations thereof; or both carbon tetrachloride and an alkene, chlorinated alkene, or combinations thereof is added to the reaction mixture. In another embodiment, at least a portion of the homogeneous liquid phase reaction mixture is treated to form a recycle product effluent stream, wherein the recycle product effluent stream contains less chlorinated alkane than the liquid phase reaction mixture. In one embodiment, the recycle product effluent stream is recycled back to the liquid phase reaction mixture

In another embodiment, at least a portion of product effluent streams (a), (c), (d) and/or (e) may be mixed with fresh material feeds before being recycled back into the reactor in batch mode or continuous mode wherein the fresh material feeds comprise the alkene, chlorinated alkene, or combinations thereof, carbon tetrachloride, the liquid free radical initiator, optional ligand, the transition metal salt or combinations thereof. In various embodiments, the recycle product effluent streams and fresh material feeds may be introduced into the reactor separately or mixed together before entering the process. The introduction of these fresh material feeds into the reactor or mixing the recycle product effluent streams with fresh materials feeds increases the efficiency of the process, reduces the overall cost, maintains the kinetics, increase the through-put, and reduces the by-products produced by the process. The amounts of the product effluent streams recycled to the reactor or fresh material feeds added to the reactor may be the same or different. One way to measure the amount of product effluent streams being recycled and/or fresh material feeds being added to the reactor is to identify the mass flow of the materials. The product effluent streams being recycled to the reactor has product effluent streams mass flow, while the fresh material feeds being added to the reactor has a fresh material feed mass flow. Mass flows may be measured using methods known in the art.

Generally, the mass ratio of the product effluent stream mass flow being recycled to the fresh material feed mass flow is adjusted to not only maintain the conversion of the process but also maintain the kinetics of the process.

In an embodiment, the recycle stream is added to the reaction mixture at rate1, the fresh feed comprising at least one of carbon tetrachloride, an alkene, chlorinated alkene, a liquid phase free radical initiator or a transition metal salt, wherein the components of the fresh feed are not recycled, is added to the reaction mixture at rate2, and the ratio of rate1 to rate2 is adjusted to maintain the reaction conversion.

In yet another embodiment, the transition metal salt may be separated from the product stream by means of extraction. This extraction, using water or another polar solvent, may remove spent or deactivated catalyst. In another embodiment, the extraction may separate the active transition metal ligand complex which may be introduced back into the second reaction vessel or other downstream processes. Using the extraction processes defined above may provide added efficiency to the process in respect to overall cost.

Product effluent streams (b) comprising chlorinated alkane produced in the process may have a yield of at least about 20%. In various embodiments, the product effluent stream (b) comprising chlorinated alkane produced in the process may have a yield of at least about 20%, at least about 50%, at least about 70%, at least about 75%, at least about 80%, at least about 85%, at least about 90%, at least about 95%, or at least about 99%.

The chlorinated alkane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (III) Preferred Embodiments: 1,1,1,3-Tetrachloropropane

(a) Process for the Preparation of 1,1,1,3-Tetrachloropropane

One aspect of the present disclosure encompasses processes for the preparation of 1,1,1,3-tetrachloropropane. The process commences by preparing a homogeneous, liquid phase reaction mixture comprising ethylene, carbon tetrachloride, a liquid free radical initiator, a transition metal salt, and a ligand. Thus, a product mixture comprising the 1,1,1,3-tetrachloropropane, heavy by-products, and light by-products is formed. The liquid phase free radical initiator is described in Section (I)(c). The transition metal salt is described in Section (I)(d). The ligand is described in Section (I)(e). In a preferred embodiment, the liquid free radical initiator is azobisisobutyronitrile, the ligand is tributylphosphate, and the transition metal salt is FeCl₃. In another preferred embodiment, the liquid free radical initiator is azobisisobutyronitrile, the ligand is butanenitrile, and the transition metal salt is CuCl₂.

(a) Reaction Conditions

The reaction conditions for the preparation of the homogeneous, liquid phase reaction mixture is described above in Section (I)(g).

(b) Output from Process

In a preferred embodiment, the produces 1,1,1,3-tetrachloropropane, light by-products, and heavy by-products. In general, the process produces 1,1,1,3-tetrachloropropane in at least 50 weight percent (wt %) in the liquid phase of the reactor. In various embodiments, 1,1,1,3-tetrachloropropane is produced in at least 50 wt %, in at least 60 wt %, in at least 70 wt %, in at least 80 wt %, in at least 90 wt %, in at least 95 wt %, or in at least 99 wt % in the liquid phase of the reactor.

In general, carbon tetrachloride is converted into 1,1,1,3-tetrachloropropane in at least 50% conversion. In various embodiments, the % conversion of carbon tetrachloride into 1,1,1,3-tetrachloropropane is at least 50%, in at least 60%, in at least 70%, in at least 80%, in at least 90%, or at least 95%.

Generally, the process produces 1,1,1,3-tetrachloropropane, light by-products and heavy by-products. These heavy by-products are produced in less than 5 weight % in the entire product distribution. In various embodiments, these heavy by-products may be less than 4 weight %, less than 3 weight %, less than 2 weight %, or less than 1 weight %.

(c) Separation of 1,1,1,3-Tetrachloropropane.

The separation of 1,1,1,3-tetrachloropropane and the recycle streams is described above in Section (II).

Product effluent streams (b) comprising the 1,1,1,3-tetrachloropropane produced in the process may have a yield of at least about 20%. In various embodiments, the product effluent streams (b) comprising 1,1,1,3-tetrachloropropane produced in the process may have a yield of at least about 30%, at least about 50%, at least about 70%, at least about 75%, at least about 80%, at least about 85%, at least about 90%, at least about 95%, or at least about 99%.

The 1,1,1,3-tetrachloropropane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (IV) Preferred Embodiments: 1,1,1,3,3-Pentachloropropane

(a) Process for the Preparation of 1,1,1,3,3-Pentachloropropane

One aspect of the present disclosure encompasses processes for the preparation of 1,1,1,3,3-pentachloropropane. The process commences by preparing a homogeneous, liquid phase reaction mixture comprising contacting vinyl chloride, carbon tetrachloride, a liquid free radical initiator, a transition metal salt, and optionally a ligand. Thus, a product mixture comprising the 1,1,1,3,3-pentachloropropane, heavy by-products, and light by-products is formed. The liquid phase free radical initiator is described in Section (I)(c). The transition metal salt is described in Section (I)(d). The ligand is described in Section (I)(e). In a preferred embodiment, the liquid free radical initiator is azobisisobutyronitrile, the ligand is tributylphosphate, and the transition metal salt is FeCl₃. In another preferred embodiment, the liquid free radical initiator is azobisisobutyronitrile, the ligand is butanenitrile, and the transition metal salt is CuCl₂.

(b) Reaction Conditions

The reaction conditions for the preparation of the homogeneous, liquid phase reaction mixture is described above in Section (I)(g).

(c) Output from Process

In a preferred embodiment, the produces 1,1,1,3,3-pentachloropropane, light by-products, and heavy by-products. In general, the process produces 1,1,1,3,3-pentachloropropane in at least 50 weight percent (wt %) in the liquid phase of the reactor. In various embodiments, 1,1,1,3,3-pentachloropropane is produced in at least 50 wt %, in at least 60 wt %, in at least 70 wt %, in at least 80 wt %, in at least 90 wt %, in at least 95 wt %, or in at least 99 wt % in the liquid phase of the reactor.

In general, carbon tetrachloride is converted into 1,1,1,3,3-pentachloropropane in at least 50% conversion. In various embodiments, the % conversion of carbon tetrachloride into 1,1,1,3,3-pentachloropropane is at least 50%, in at least 60%, in at least 70%, in at least 80%, in at least 90%, or at least 95%.

Generally, the process produces 1,1,1,3,3-pentachloropropane, light by-products, and heavy by-products. These heavy by-products are produced in less than 5 weight % in the entire product distribution. In various embodiments, these heavy by-products may be less than 4 weight %, less than 3 weight %, less than 2 weight %, or less than 1 weight %.

(d) Separation of 1,1,1,3,3-Pentachloropropane.

The separation of 1,1,1,3,3-pentachloropropane and the recycle streams is described above in Section (II).

Product effluent stream (b) comprising the 1,1,1,3,3-pentachloropropane produced in the process may have a yield of at least about 20%. In various embodiments, the product effluent stream (b) comprising 1,1,1,3,3-pentachloropropane produced in the process may have a yield of at least about 30%, at least about 50%, at least about 70%, at least about 75%, at least about 80%, at least about 85%, at least about 90%, at least about 95%, or at least about 99%.

The 1,1,1,3,3-pentachloropropane contained in product effluent stream (b) from the process may have a weight percent at least about 50%, at least about 60%, at least about 65%, at least about 70%, at least about 75%, at least about 80%, at least about 85%, at least about 90%, at least about 95%, at least about 99%, at least about 99.5%, or at least about 99.9%.

### (V) Further Reaction of the Halogenated Alkanes

In one aspect, disclosed herein are processes for the conversion of chlorinated alkanes, such as 1,1,1,3-tetrachloropropane or 1,1,1,3,3-pentachloropropane to one or more hydrofluoroolefins. These processes comprise contacting the chlorinated alkanes with a fluorinating agent in the presence of a fluorination catalyst, in a single reaction or two or more reactions. These processes can be conducted in either gas phase or liquid phase with the gas phase being preferred at temperatures ranging from 50° C. to 400° C.

Generally, a wide variety of fluorinating agents can be used. Non-limiting examples of fluorinating agents include HF, F₂, CIF, AlF₃, KF, NaF, SbF₃, SbF₅, SF₄, or combinations thereof. The skilled artisan can readily determine the appropriate fluorination agent and catalyst. Examples of hydrofluoroolefins that may be produced utilizing these processes include, but are not limited to 2,3,3,3-tetrafluoroprop-1-ene (HFO-1234yf), 1,3,3,3-tetrafluoroprop-1-ene (HFO-1234ze), 3,3,3-trifluoroprop-1-ene (HFO-1243zf), and 1-chloro-3,3,3-trifluoroprop-1-ene (HFCO-1233zd).

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “Tet” refers to carbon tetrachloride.

The term “TBP” refers to tributylphosphate.

The term “AIBN” refers to azobisisobutyronitrile.

The term “250FB” refers to 1,1,1,3-tetrachloropropane.

Having described the invention in detail, it will be apparent that modifications and variations are possible without departing from the scope of the invention defined in the appended claims.

### EXAMPLES

The following examples illustrate various embodiments of the invention.

### Example 1: Preparation of 1,1,1,3-Tetrachloropropane (Denoted as Run K)

17.6 g Tet, 0.38 g TBP and 0.076 g FeCl₃ were added to an autoclave constructed of Hastelloy B and equipped with means for heating and stirring. The autoclave was sealed and stirring was initiated. Ethylene was fed into the reactor to a pressure of 120 psig and then the reactor was heated to 110° C. Ethylene feed was added to maintain pressure. After 1 hour, a sample was drawn and analyzed by gas chromatography.

### Example 2: Preparation of 1,1,1,3-Tetrachloropropane (Denoted as Run 2L)

17.6 g Tet, 0.26 g TBP and 0.058 g FeCl₂ were added to the autoclave as described in Example 1. The conditions of Example 1 were repeated, except that samples were drawn at 1 and 2.1 hours which were analyzed by gas chromatography.

### Example 3: Preparation of 1,1,1,3-Tetrachloropropane (Denoted as Run 30)

16 g Tet, 0.014 g TBP, 0.0079 g FeCl₃, and 0.20 g AIBN were added to the autoclave as described in Example 1. The autoclave was sealed and stirring was initiated. Ethylene was fed into the autoclave to a pressure of 120 psig, and then the autoclave was heated to 60° C. Ethylene feed was added to maintain pressure. A sample was drawn at 1.4 hours and the temperature was increased to 80° C. A sample was drawn at 2.7 hours and the temperature was increased to 100° C. A final sample was drawn at 3.95 hours, and all samples were analyzed by gas chromatography.

### Example 4: Preparation of 1,1,1,3-Tetrachloropropane (Denoted as Run 3R)

16 g Tet, 0.028 g TBP, 0.016 g FeCl₃, and 0.17 g AIBN were added to the autoclave as described in Example 1. The autoclave was sealed and stirring was initiated. Ethylene was fed into the autoclave to a pressure of 120 psig and then heated to 80° C. Ethylene feed was added to the autoclave to maintain pressure. A sample was drawn at 1.4 hours and the temperature was increased to 80° C. Samples were drawn at 1.5, 3.25 and 4.8 hours. All samples were analyzed by gas chromatography.

### Results from the Above Examples

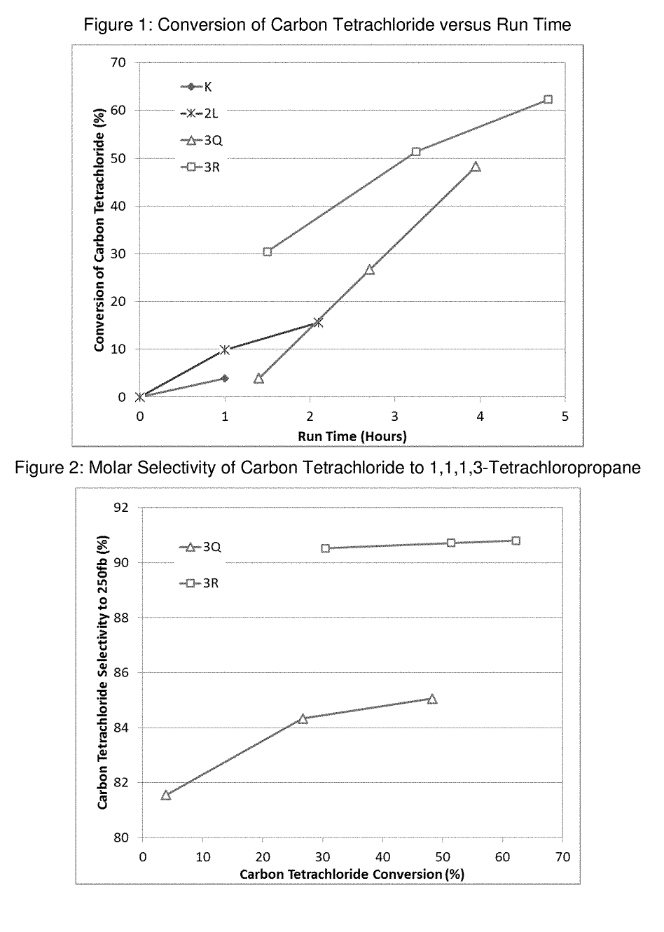

FIG. 1 shows that the activity of the homogenous Fe(II) or Fe(III)-TBP system is low when solid metallic iron is not present. The activity improved and conversion was higher when the initiator AIBN was combined with the homogenous Fe(II) or Fe(III)-TBP system, even though the temperature was lower and no solid metallic iron was present.

FIG. 2 shows that adding additional FeCl₃ to the homogenous Fe-TBP/AIBN catalyst system increases the selective formation of 250(FB). Additional TBP was also added to keep the iron in solution. In both Examples 3 and 4, there was only a small molar excess of TBP over iron.

What is claimed is:

1. A process for preparing chlorinated alkanes, the process comprising:
a) preparing a homogeneous, liquid phase reaction mixture comprising carbon tetrachloride (Tet), an alkene, chlorinated alkene, or combinations thereof, a liquid phase free radical initiator and a transition metal salt; and
b) forming a product mixture comprising light by-products, heavy by-products, and the chlorinated alkane.

2. The process of claim 1, wherein the alkene comprises ethylene.

3. The process of claim 1, wherein the chlorinated alkene comprises vinyl chloride monomer.

4. The process of claim 1, wherein the transition metal salt comprises an aluminum salt, a bismuth salt, a chromium salt, a cobalt salt, a copper salt, a gallium salt, a gold salt, an indium salt, an iron salt, a lead salt, a magnesium salt, a manganese salt, a mercury salt, a nickel salt, a platinum salt, a palladium salt, a rhodium salt, a samarium salt, a scandium salt, a silver salt, a titanium salt, a tin salt, a zinc salt, a zirconium salt, or combinations thereof.

5. The process of claim 1, wherein the metal salt comprises an iron salt.

6. The process of claim 5, wherein the iron salt comprises iron (II) chloride, iron (Ill) chloride, or combinations thereof.

7. The process of claim 1, wherein the transition metal catalyst further comprises at least one ligand.

8. The process of claim 7, wherein the at least one ligand comprises a trialkylphosphate, a trialkylphosphite, a C₃-C₆ alkanenitrile, or combinations thereof.

9. The process of claim 7, wherein the trialkylphosphate comprises triethylphosphate, tripropylphosphate, triisopropylphosphate, tributylphosphate, or combinations thereof.

10. The process of claim 1, wherein the free radical initiator comprises an azo compound, a peroxide, ultraviolet light, or combinations thereof.

11. The process of claim 1, wherein after at least some chlorinated alkane is formed, fresh feed comprising carbon tetrachloride; an alkene, chlorinated alkene, or combinations thereof; or both carbon tetrachloride and an alkene, chlorinated alkene, or combinations thereof is added to the reaction mixture.

12. The process of claim 1, wherein at least a portion of the homogeneous liquid phase reaction mixture is treated to form a recycle product effluent stream, wherein the recycle product effluent stream contains less chlorinated alkane than the liquid phase reaction mixture.

13. The process of claim 12, wherein the recycle product effluent stream is recycled back to the liquid phase reaction mixture.

14. The process of claim 1, wherein the homogeneous liquid phase reaction mixture is stirred and stirring the homogeneous liquid phase reaction mixture comprises mechanical stirring, stirring by liquid jet mixing, or combinations thereof.

15. The process of claim 1, wherein the homogeneous liquid phase reaction mixture is formed in a reactor and the reactor comprises a draft tube.

16. The process of claim 13, wherein the recycle stream is added to the reaction mixture at rate1, the fresh feed comprising at least one of carbon tetrachloride, an alkene, chlorinated alkene, a liquid phase free radical initiator or a transition metal salt, wherein the components of the fresh feed are not recycled, is added to the reaction mixture at rate2, and the ratio of rate1 to rate2 is adjusted to maintain the reaction conversion.

17. The process of claim 1, wherein the weight % of the chlorinated alkane is at least 50 weight % in the reaction mixture.

18. The process of claim 1, wherein at least 50% of the carbon tetrachloride is converted to the chlorinated alkane.

19. A process of claim 1, the process comprising:
a) preparing a reaction mixture comprising carbon tetrachloride (Tet), ethylene, FeCl₃, tributylphosphate, and a liquid free radical initiator selected from the group consisting of AIBN, peroxide, and UV light; and
b) forming a product mixture comprising light by-products, heavy by-products, and 1,1,1,3-tetrachloropropane.

20. A process of claim 1, the process comprising:
a) preparing a reaction mixture comprising carbon tetrachloride (Tet), vinyl chloride monomer, FeCl₃, tributylphosphate, and a liquid free radical initiator selected from the group consisting of AIBN, peroxide, and UV light; and
b) forming a product mixture comprising light by-products, heavy by-products, and 1,1,1,3,3-pentachloropropane.
